(19) United States
(12) Patent Application Publication
(10) Pub. No.: US 2023/0233613 A1
(43) Pub. Date: Jul. 27, 2023

(54) PHARMACEUTICAL COMPOSITION FOR TREATING CHRONIC STROKE

(71) Applicant: Gwo Xi Stem Cell Applied Technology Co., Ltd., Zhubei City (TW)

(72) Inventors: Po-Cheng Lin, Zhubei City (TW); Pi-Chun Huang, Zhubei City (TW); Chia-Hsin Lee, Zhubei City (TW); Ming-Hsi Chuang, Zhubei City (TW); Chun-Hung Chen, Zhubei City (TW); Chao-Liang Chang, Zhubei City (TW); Kai-Ling Zhang, Zhubei City (TW); Yi-Chun Lin, Zhubei City (TW); Yu-Chen Tsai, Zhubei City (TW); Peggy Leh Jiunn Wong, Zhubei City (TW); Ruei-Yue Liang, Zhubei City (TW)

(21) Appl. No.: 17/963,219

(22) Filed: Oct. 11, 2022

Related U.S. Application Data
(60) Provisional application No. 63/302,595, filed on Jan. 25, 2022.

(51) Int. Cl. A61K 35/28; A61K 9/00; C12N 5/0789

(52) U.S. Cl. CPC A61K 35/28; A61K 9/0085; A61K 9/0019; C12N 5/0647; C12N 2501/10; C12N 2502/1382; C12N 2501/2304; C12N 2501/2313

(57) ABSTRACT

The present invention provides a pharmaceutical composition for treating chronic stroke, involving injection via brain into the cranium of a patient having chronic stroke for six months or more; the pharmaceutical composition is a suspension at least comprising TS stem cells, an active synergistic component and a growth factor, wherein the expression level of CD34 and CD45 of the TS stem cells is 10% or less, and the expression level of CD90 and CD105 is 90% or more; the active synergistic component is an extracellular vesicle; the growth factor is at least one selected from the group consisting of HGF, G-CSF, Fractalkine, IP-10, EGF, IL-1α, IL-1β, IL-4, IL-5, IL-13, IFNγ, TGFα and sCD40L. The present invention overcomes the limitations of previous cell therapy and provides a cell-based preparation that is clinically safe and therapeutically effective for chronic cerebral stroke.

### CROSS-REFERENCE TO RELATED APPLICATIONS

The present application claims the benefit of U.S. Provisional Patent Application No. 63/302,595 filed on Jan. 25, 2022, which is incorporated by reference herein in its entirety.

### STATEMENT REGARDING PRIOR DISCLOSURES BY AN INVENTOR OR JOINT INVENTOR

This invention was the subject of a presentation to the public by inventor on Oct. 13, 2021 entitled “Intracerebral transplantation of autologous adipose-derived stem cells for chronic ischemic stroke: A phase I study” in Journal of Tissue Engineering and Regenerative Medicine, 1-11. https://doi.org/10.1002/term.3256.

### BACKGROUND OF THE INVENTION

### 1. Field of the Invention

The present invention relates to a pharmaceutical composition, in particular to a pharmaceutical composition for treating chronic stroke.

### 2. Description of the Related Art

Stroke is a debilitating disease described as loss of brain function with symptoms lasting 24 hours or longer or leading to death. Stroke involving the middle cerebral artery can result in significant mortality and morbidity due to irreversible neuronal damage. In 2020, the World Health Organization (WHO) reported that stroke caused 7.8 million deaths worldwide and was one of the leading causes of disability in adults.

Cerebral stroke can be divided into acute stroke, subacute stroke and chronic stroke according to onset time. Acute stroke refers to the initial stage after the onset of cerebral stroke (within 3 months of onset), subacute stroke refers to the stage after discharge from hospital in the acute stroke (3-6 months of onset), and then all are classified as chronic stroke (6 months or more of onset). Various therapies have been developed in the past to treat acute stroke. Traditionally, one of the common methods of treating acute stroke is conducted by thrombolytic therapy. However, studies seem to indicate that thrombolytic therapy has not achieved much success in improving the overall health of patients with acute stroke. In addition, there is currently no active treatment available to restore neurological function in most patients with stroke more than 6 months. Within three months after stroke, it is the golden treatment period of rehabilitation, at which time the functional recovery is the fastest and the rehabilitation effect is the best. Three to six months after stroke, the rate of functional recovery is significantly reduced. After six months of stroke, the recovery of function gradually ceases, at which time there is a very limited space for rehabilitation progress, and the goal of rehabilitation is to maintain existing functions.

The study of the mesenchymal stem cell (MSC) has attracted great interest of researchers and clinicians due to its ability to differentiate into neuron-like cells and immunomodulatory properties. Mesenchymal stem cells may also help reduce cerebral edema and accelerate recovery after acute stroke because the blood-brain barrier (BBB) is semi-permeable during acute stroke, which allows mesenchymal stem cells to be administered intravenously (IV) and then homing into the brain by signaling through the area of brain damage, so that mesenchymal stem cell transplantation can improve acute stroke. However, after three months of stroke, the blood-brain barrier has been shut down and a phase of chronic stroke is entered, at which time stem cells administered intravenously cannot enter the brain and the associated growth factor signals are no longer generated, therefore the purpose of treatment cannot be achieved.

Furthermore, there are several different cell transplantation routes in previous clinical studies, such as intravenous or intra-arterial (IA) injection. The advantage of intravenous administration is that this route is considered non-invasive and can be carried out easily. However, some preclinical studies suggest that stem cells may be cleared by the lung and liver during circulation, and that the ability of stem cells to cross the blood-brain barrier may be limited; in contrast to intravenous administration, stem cells can be transferred directly into the brain by intra-arterial administration. Nevertheless, there is concern that intra-arterial administration may lead to cell aggregation, resulting in some microthrombosis and possibly further injury.

In conclusion, in the late stage of treatment of cerebral stroke, it is necessary to overcome the mechanisms that allow cells to enter the damaged area of the brain and to effectively survive and reinitiate brain repair, and at the same time, safer, less invasive and clinically effective alternative methods of treatment of cerebral stroke become the focus of medical experts. However, currently known cellular drugs are mostly designed for intravenous administration. Thus, there remains a need for new cell-based compositions that are clinically safe and therapeutically effective for treating human cerebral stroke.

### SUMMARY OF THE INVENTION

In order to overcome the above limitations, the present invention designs a preparation comprising stem cells and extracellular vesicles produced under specific conditions and growth factors, and a dosage form already available for intracranial injection overcomes the limitations of previous cell therapies, and a cell-based preparation and its use are provided, which is clinically safe and therapeutically effective for chronic cerebral stroke.

In particular, the present invention can provide a pharmaceutical composition for treating chronic stroke involving injection via brain into the cranium of a patient having chronic stroke for six months or more, wherein the pharmaceutical composition is a suspension at least comprising TS stem cells, an active synergistic component and a growth factor; and wherein the expression level of CD34 and CD45 of the TS stem cells is 10% or less, and the expression level of CD90 and CD105 is 90% or more; the active synergistic component is an extracellular vesicle; and the growth factor is at least one selected from the group consisting of HGF, G-CSF, Fractalkine, IP-10, EGF, IL-1α, IL-1β, IL-4, IL-5, IL-13, IFNγ, TGFα and sCD40L.

According to one embodiment of the present invention, in the pharmaceutical composition, the amount of the TS stem cells is at least 1×10⁷/mL, the amount of the active synergistic component is 7×10¹¹˜1.5×10¹³/mL, and the amount of the growth factor is 0.01˜4,000 pg/mL.

According to one embodiment of the present invention, the patient has a score of National Institutes of Health Stroke Scale (NIHSS) between 8 and 30.

According to one embodiment of the present invention, the TS stem cells are obtained by culturing adipose-derived stem cells in an expansion medium and the initial culture density of the adipose-derived stem cells is 5,000˜15,000 stem cells/cm²; and the expansion medium is Keratinocyte-SFM medium containing 1-100 mMof N-acetyl-L-cysteine, 0.05-50 mMof L-ascorbic acid 2-phosphate.

According to one embodiment of the present invention, the expansion medium is placed on a culture plate made of a material containing at least 20% or more oxygen-containing functional groups.

According to one embodiment of the present invention, the pharmaceutical composition is obtained by allowing the TS stem cells to stand in water for injection at a temperature of 2-10° C. for 1-24 hours, and the TS stem cells release the active synergistic component and the growth factor during standing, wherein the water for injection can be selected from any one of the groups consisted of distilled water for injection, physiological saline injection, 0.45%˜3% sodium chloride injection, 2.5%˜50% glucose injection, Lactated Ringer's B injection and Ringer's Solution.

According to one embodiment of the present invention, the source of adipose-derived stem cells is autologous or allogeneic.

According to one embodiment of the present invention, an endotoxin test result of the TS stem cells is less than 0.06 EU/mL.

According to one embodiment of the present invention, a Mycoplasma test result of the TS stem cells is no reaction.

According to one embodiment of the present invention, the cell viabilityTS stem cells is at least 80% or more.

According to one embodiment of the present invention, the particle size of the active synergistic component is 30 nm to 1 μm, and the active synergistic component express ALIX, TSG101, CD9 and CD81.

According to one embodiment of the present invention, the amount of HGF is 2,000˜4,000 pg/ml.

According to one embodiment of the present invention, the amount of G-CSF is 200˜400 pg/ml.

According to one embodiment of the present invention, the amount of TGFα is 0.01˜0.2 pg/ml.

According to one embodiment of the present invention, the amount of IL-4 is 10˜20 pg/ml.

According to one embodiment of the present invention, the amount of IL-13 is 2-3 pg/ml.

### BRIEF DESCRIPTION OF THE DRAWINGS

FIG. 1 is a graph showing cell growth curves of Examples 1 to 4.

FIGS. 2A-2D are graphs showing the change in NIHSS, Barthel Index assessment, Berg Balance Test and Fugl-Meyer Assessment (FMA) for subjects 17B001, 17B002, 17B003 sequentially.

FIG. 3 shows T2-FLAIR images of subjects. In the graph, (a), (b) are images of subject 17B001, (c), (d) are images of subject 17B002, and (e), (f) are images of subject 17B003. In the graph, the left column of (a), (c) and (e) shows the T2-FLAIR image before injection; the left column of (b), (d) and (f) shows T2 images before injection (white arrows indicate the area of infarction); the middle column of the graph shows the CT scan image detected immediately after injection (white arrow indicates one of the three injection sites); in the graph, the right column of (a), (c) and (e) shows the T2 image detected 2 weeks after injection; and the right column of (b), (d) and (f) shows the T2 images detected 6 months after injection.

### DETAILED DESCRIPTION OF THE PREFERRED EMBODIMENTS

In order that the objects, features and advantages of the present invention will become more fully apparent to those skilled in the art and the present invention can be carried out, the technical features and embodiments of the present invention are specifically illustrated herein with reference to the accompanying drawings and the preferred examples are listed to further illustrate. In the following description, the drawings referred to are schematic representations relating to features of the invention, and the drawings are not complete or not required to be complete according to actual situations.

As used herein, all technical and scientific terms used herein have the same meaning as commonly understood by those skilled in the art. Furthermore, as used herein, singular terms shall include plural forms and plural terms shall include singular formsr, unless otherwise expressly contradicted by context.

Although the numerical ranges and parameters used to define the broad scope of the invention are approximate numerical values, the numerical values set forth in the specific examples are reported as precisely as possible herein. However, any numerical value essentially inevitably contains the standard deviation found in the respective testing method. As used herein, “about” generally means that the actual value is within plus or minus 10%, 5%, 1%, or 0.5% of a particular value or range. Alternatively, the term “about” means that the actual numerical value falls within an acceptable standard error of the mean value, which depends on the consideration of those skilled in the art with common knowledge in the technical field to which the invention belongs. Except the examples, or unless explicitly stated otherwise, all ranges, amounts, values and percentages used herein (e.g. to describe amounts of material, amounts of time, temperature, operating conditions, proportions of amounts, and the like) are to be understood as modified by the word “about”. Accordingly, unless indicated to the contrary, the numerical parameters set forth in the present specification and attached claims are approximate numerical values that may vary depending upon the requirements. At the very least, these numerical parameters should be understood to mean the number of significant digits indicated and the number obtained by applying the general carry method.

In order that the description of the present disclosure may be more thorough and complete, an illustrative description of mode of embodiments and specific examples of the invention is set forth below. However, this is not the only form in which the specific examples of the invention may be practiced or utilized. The embodiments encompass the features of many specific examples and the method steps and their order to construct and operate these examples. However, other specific examples may be utilized to achieve the same or equivalent functions and step sequences.

Next, the present invention will be described with reference to specific examples.

### Examples 1-4

The cells used in these examples were Human Adipose-derived Stem Cells (hADSCs).

2-5 g of adipose tissue is collected from subcutaneous adipose in the abdominal wall by performing liposuction from healthy donors during abdominal surgery, and the operation time for adipose extraction is about 1 hour or less, and the wound is less than 1 cm. All donors provided informed consent form. Human adipose tissue was placed in Ca²⁺/Mg²⁺ free phosphate buffer solution (PBS) and immediately transferred to the laboratory.

Human adipose tissue was removed from the medium for transport and placed in a culture plate, washed 3 to 4 times with Ca²⁺/Mg²⁺ free phosphate buffered solution (PBS), and cut into small pieces (volume about 1-3 mm³). The tissue was dissociated with 0.1-0.3% of collagenase for 60 minutes at 36.5-38.5° C. After digestion of collagenase, cells and undigested tissue fragments were separated from the granules of stromal vascular fractions (SVF) by centrifugation at 500 g for 5-15 min at 20-25° C., and dissociated cells were collected and cultured at 36.5-38.5° C. in an incubator supplied with 5% CO₂. After 1-2 days of culture, the supernatant and fragments were removed from the culture to obtain primary adipose-derived stem cells.

Then, the primary adipose-derived stem cells were cultured and expanded in different medium, and the medium used in each example were as follows:

Example 1: 0.5×10⁵ adipose-derived stem cells were cultured in 6-well cell culture dishes (Corning) in Keratinocyte-SFM medium (Gibco) containing 1-100 mM of N-acetyl-L-cysteine (Sigma), 0.05-50 mM of L-ascorbic acid 2-phosphate (sigma) for 1, 4 and 7 days, and the culture environment is in a cell incubator controlled at a temperature of 36.5-38.5° C. and with 5% carbon dioxide.

Example 2: 0.5×10⁵ adipose-derived stem cells were cultured in 6-well cell culture dishes (Corning) in DMEM/F12 medium (Gibco) containing 1-10 mg/ml of Human Serum Albumin (Bio-Pure), 0.05-50 mM of L-ascorbic acid 2-phosphate (sigma), 1 mM-40 mM of Sodium bicarbonate (Sigma) for 1, 4 and 7 days, and the culture environment is in a cell incubator controlled at a temperature of 36.5-38.5° C. and with 5% carbon dioxide.

Example 3: 0.5×10⁵ adipose-derived stem cells were cultured in 6-well cell culture dishes (Corning) in DMEM/F12 medium (Gibco) containing 1-10 mg/ml of Human Serum Albumin (Bio-Pure), 0.05-50 mM of L-ascorbic acid 2-phosphate (sigma), 1 mM-40 mM of Sodium bicarbonate (Sigma), 5-15 mM of HEPES (Sigma) for 1, 4, 7 days, and the culture environment is in a cell incubator controlled at a temperature of 36.5-38.5° C. and with 5% carbon dioxide.

Example 4: 0.5×10⁵ adipose-derived stem cells were cultured in 6-well cell culture dishes (Corning) in DMEM/F12 medium (Gibco) containing 5-20 wt % fetal bovine serum (Hyclone) for 1, 4 and 7 days, and the culture environment is in a cell incubator controlled at a temperature of 36.5-38.5° C. and with 5% carbon dioxide.

Cell survival rate (Cell viability) was assessed with a ADAM-MC™ Automatic Cell counter (Digital Bio, NanoEnTek Inc.).

Each example was analyzed for cell viability and number of cells on the culture day of day 1, day 4, day 7, respectively, and the results are reported in Table 1.

TABLE 1

| | | Example 1 | Example 2 | Example 3 | Example 4 |
|---|---|---|---|---|---|
| Cell | Day 1 | 67 | 92 | 90 | 74 |
| viability | Day 4 | 88 | 98 | 98 | 98 |
| (%) | Day 7 | 89 | 98 | 96 | 97 |
| Number | Day 1 | 0.457 × 10⁵ | 0.369 × 10⁵ | 0.432 × 10⁵ | 0.336 × 10⁵ |
| of cells | Day 4 | 5.534 × 10⁵ | 3.460 × 10⁵ | 4.174 × 10⁵ | 1.548 × 10⁵ |
| (num- | Day 7 | 8.765 × 10⁵ | 6.808 × 10⁵ | 7.904 × 10⁵ | 1.860 × 10⁵ |
| ber) | | | | | |

In addition, the adipose-derived stem cells obtained in each example were analyzed, respectively, for the expression level of surface antigen on the culture day of day 7.1×10⁶/mL adipose-derived stem cells at 100 μL were taken into a microcentrifuge tube, and fluorescently labeled CD73, CD90, CD105, CD14, CD19, CD34, CD45 and HLA-DR (Becton Dickinson) antibodies were added in a ratio of 1:100 and mixed well, and allowed to stand in the dark, and then the cell markers were analyzed using a BD AccuriC6 flow cytometer (Becton Dickinson), and the results were recorded in Table 2 after the analysis was completed.

TABLE 2

| | | Example 1 | Example 2 | Example 3 |
|---|---|---|---|---|
| Expression level | CD 73 | 99.96 | 99.81 | 99.66 |
| of surface | CD 90 | 100 | 99.98 | 99.94 |
| antigenlevel(%) | CD 105 | 99.19 | 99.69 | 98.17 |
| | CD 14 | 0.06 | 0.07 | 0.15 |
| | CD 19 | 0.06 | 0.17 | 0.13 |
| | CD 34 | 0.07 | 0.29 | 0.23 |
| | CD 45 | 0.10 | 0.16 | 0.18 |
| | HLA-DR | 0.04 | 0.07 | 0.12 |

As can be seen from the above results, in the cell viability results, the cell viability of Example 2 and Example 3 were both 95% or more on day 7, while the cell viability of Example 1 (KSFM) was about 90%. In the results of cell viability, the cell viability of the cells cultured in Examples 1-4 was about 90% at day 7.

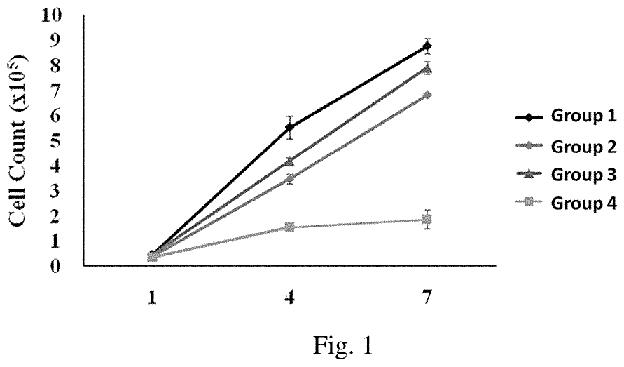

Next, referring to FIG. 1, which is a graph showing the cell growth curves of Examples 1-4, the results in FIG. 1 show that the number of cells of Example 1 is the most regardless of day 4 or day 7, followed by Example 3, Example 2, and Example 4, wherein the number of cells of Example 4 is significantly less than the other three groups.

Also, the surface antigens of each example were confirmed, ADSCs cultured in Examples 1-3 expressed specific mesenchymal stem cell markers CD73, CD90 and CD105 at high levels, while hematopoietic cell markers CD14, CD19, CD34, CD45 and HLA-DR molecules expressed at very low levels, consistent with the characteristics of adipose-derived stem cells.

In addition, the doubling-time of the cells after cultured to day 7 in Examples 1-4 were compared, and the results are shown in Table 3.

TABLE 3

| | Example 1 | Example 2 | Example 3 | Example 4 |
|---|---|---|---|---|
| Doubling-time (hours) | 20.01 | 22.30 | 22.00 | 32.67 |
| Doubling-time | 38.8% | 31.7% | 32.7% | — |
| (the result of | shorter | shorter | shorter | |
| example 4 as 100%) | | | | |

The doubling-time for Example 1 was 20.01 hours, while the doubling-time for Examples 2-4 was 22.30 hours, 22.00 hours and 32.67 hours respectively.

From the above results, it can be seen that the medium used in Example 1 is more effective in increasing the expanded number of adipose-derived stem cells than the medium used in the other examples, without affecting the cell viability and the characteristics of surface antigen.

### Examples 5, 6

In the examples, the primary adipose-derived stem cells obtained in the same manner as in the above-described Examples 1-4 were subjected to cell expansion on a culture plate made of a material containing an oxygen-containing functional group proportion of 20% or more and on a culture plate made of a material containing an oxygen-containing functional group proportion of less than 20%, wherein the outer material of the culture plate made of a material containing an oxygen-containing functional group proportion of 20% or more is polystyrene, due to oxygen-containing functional groups are incorporated on the surface of the polystyrene, the culture surface has a net negative surface charge, which raises the surface of the culture plate more hydrophilic and wet, and facilitates cell attachment and growth. The culture mode of each example is as follows:

Example 5: 1×10⁶ adipose-derived stem cells were cultured in the same medium composition as in Example 1 in a culture plate (HYPERFlask, Corning) made of a material containing an oxygen-containing functional group proportion of 20% or more to eighty percent full (about 10-14 days, the number of cells is about 7×10⁷-3.146×10⁸), and the culture environment is in a cell incubator controlled at a temperature of 36.5-38.5° C. and with 5% carbon dioxide.

Example 6: 1×10⁶ adipose-derived stem cells were cultured in the same medium composition as in Example 1 in a culture plate (175T Flask, Corning) made of a material containing an oxygen-containing functional group proportion of less than 20% to eighty percent full (about 10-14 days), and the culture environment is in a cell incubator controlled at a temperature of 36.5-38.5° C. and with 5% carbon dioxide.

Each example was analyzed respectively for number of cells and cell survival rate, and degree of surface antigen expression at the time of culture to eighty percent full (about 10-14 days), and the results were recorded in Table 4.

TABLE 4

| | | Example 5 | Example 6 |
|---|---|---|---|
| Number of cells (number) | | 2.56 × 10⁸ | 1.64 × 10⁸ |
| Cell survival rate | | 95% | 96% |
| Degree of surface antigen | CD 29 | 99.99 | 99.95 |
| expression (%) | CD44 | 99.96 | 99.96 |
| | CD 90 | 100 | 99.98 |
| | CD 105 | 97.82 | 97.13 |
| | CD 14 | 0.00 | 0.02 |
| | CD 34 | 0.03 | 0.01 |
| | CD 45 | 0.02 | 0.03 |
| | HLA-DR | 0.13 | 0.15 |

As can be seen from the results in Table 4 above, the number of cells of Example 5 increased from 1×10⁶ cells to 2.56×10⁸ cells; the number of cells for Example 6 ranged from 1×10⁶ cells to 1.64×10⁸ cells. It can be seen that in the case of culturing the adipose-derived stem cells on a culture plate made of a material containing an oxygen-containing functional group proportion of 20% or more in the same medium composition, the expansion rate is significantly higher than that in a culture plate made of a material containing an oxygen-containing functional group proportion of less than 20%.

Also, the surface antigens of each example were confirmed, ADSCs cultured in Examples 5 and 6 expressed specific mesenchymal stem cell markers CD90 and CD105 at high levels, while hematopoietic cell markers CD14, CD34, CD45 and HLA-DR molecules expressed at very low levels, consistent with the characteristics of adipose-derived stem cells.

Cluster of Differentiation (CD) refers to cell surface markers that cells of different lineages display or disappear at different stages of normal differentiation maturation and during activation. CD markers are protein complex or glycoprotein on cell membranes. CD markers has many uses, it is commonly used as important receptors or ligands of cells, at the same time, they can be used as surface markers for the identification and isolation of cells, and are widely involved in various stages of cells, including cell growth, cell differentiation, cell migration, etc.; Wherein CD29 (integrin 01) is known to be involved in differentiation, migration, proliferation, wound repair, tissue development and organogenesis. CD44 (hyaluronate) is expressed in stem cells and in the cells in the niches surrounding, including inflammatory cells, suggesting that CD44 plays an important role in the repair process of ischemic areas, especially stroke, in neuroinflammatory condition. CD73 (5′ ecto-nucleotidase) is a regulator of inflammatory and immune functions in the brain. CD90 (Thy-1) plays a key role in adipose-derived stem cell proliferation and metabolism by activating AKT and Cyclin D1. CD105 (endoglin) can be a relative specific marker of adipose-derived stem cells; and studies have shown that adipose-derived stem cells (CD105+ADSCs) which express CD105 have a higher growth rate and differentiation capacity. Adipose-derived stem cells highly expressing CD90 and CD105 can release neurotrophins such as: brain-derived neurotrophic factors (BDNF), neurotrophin-3 (NT3) and neurotrophin-4 (NT4) and thus prevent epilepsy.

From the above results, it can be seen that the culture plate used in Example 5 can more effectively elevate the expanded amount of adipose-derived stem cells than the culture plate used in Example 6, without affecting the cell viability and the characteristics of surface antigen.

### Examples 7, 8

In the examples, the primary adipose-derived stem cells obtained in the same manner as in the above-described Examples 1-4 were used for cell expansion, and the culture manner of each example was as follows:

Example 7: about 1×10⁷ adipose-derived stem cells were cultured in the same medium composition as in Example 1 on a culture plate made of a material containing an oxygen-containing functional group proportion of 20% or more for 7 days, and the culture environment is in a cell incubator controlled at a temperature of 36.5-38.5° C. and with 5% carbon dioxide.

Example 8: 1×10⁷ adipose-derived stem cells were cultured in the same medium composition as in Example 3 in a culture plate made of a material containing an oxygen-containing functional group proportion of 20% or more for 7 days, and the culture environment is in a cell incubator controlled at a temperature of 36.5-38.5° C. and with 5% carbon dioxide.

At the time of culture to day 7, the number of cells of each example was analyzed respectively, and it was found that the number of cells of Example 7 was increased from 1×10⁷ cells to 7.88×10⁷ cells, while the number of cells of Example 8 was decreased from 1×10⁷ cells to 1.96×10⁶ cells. It can be seen from this result that not all the culture medium can be in combination with the culture plates made of a material containing an oxygen-containing functional group proportion of 20% or more to expand the number of adipose-derived stem cells, but the culture medium in Example 1 can be used in combination with a culture plate made of a material containing tan oxygen-containing functional proportion of 20% or more to most efficiently expand the number of adipose-derived stem cells.

### Example 9

### Analysis of Extracellular Vesicles

Extracellular vesicles (EV) are heterogeneous particles, formed by outward budding or exocytosis of primitive cells, which have no functional nuclei and is unable to replication. Extracellular vesicles include exosome and microvesicles. Extracellular vesicles range in diameter from 30 nm to 1 μm. Extracellular vesicles are lipid bilayer membrane enveloping cell-derived particles containing proteins, lipids, nucleic acids, etc. Extracellular vesicles are usually rich in four tetraspanin proteins on the cell surface, mainly CD9, CD63 and CD81 and other proteins such as: ALG-2 interacting protein-X (Alix) and tumor susceptibility gene 101 (TSG101). Alix, also known as programmed cell death 6 interacting protein (PDCD6IP), is an adaptor protein that binds to ESCRT and endophilin-A proteins. Alix is expressed in neurons and is concentrated at the synapses during epileptic seizures. TSG101 and signal-transducing adaptor molecules play a key role in exosome biogenesis and secretion. It has been found that large expression of TSG101 in neural stem cells can increase exosome biogenesis related genes and thus enhance exosome secretion. Extracellular vesicles can promote intercellular communication, including crossing the blood-brain barrier. Studies have shown that in the case of stroke, certain cells preferentially release extracellular vesicles, some of which provide some degree of neuroprotection.

1×10⁸ adipose-derived stem cells obtained by expansion in Example 7 were mixed with physiological saline, and extracellular vesicles in the fluid was analyzed after standing for 1, 2, 4, 8, 16, 24, 36 and 48 hours at 2-10° C.

Following the above, the supernatant was removed to a new tube, centrifuged at 4000 g for 20 minutes, and the supernatant was again removed and filtered through a 0.22 μm filter (Merck Millipore, Billerica, Mass., USA) to remove large vesicles. Amicon Ultra-15 with a molecular weight over 100 kDa (Millipore) was used to remove free protein, and then the supernatant containing extracellular vesicles was obtained after centrifugation at an acceleration rate of 4000 g, and the solution was analyzed for extracellular vesicle concentration and particle size, and the results are recorded in Table 5.

Table 5

TABLE 5

| | | | Extracellular vesicles | | |
|---|---|---|---|---|---|
| Standing | Number | | Particle | Concen- | Concen- |
| time | (number) | Saline | size | trations | trations |
| (hour) | of cells | (ml) | (nm) | (particles/ml) | (particles/ml) |
| 1 | 1 × 10⁸ | 1 | 77.3 ± 1.3 | 1.17 × 10¹² | 9.59 × 10³ |
| 2 | 1 × 10⁸ | 1 | 76.2 ± 1.3 | 9.53 × 10¹¹ | 1.33 × 10⁴ |
| 4 | 1 × 10⁸ | 1 | 91.5 ± 1.7 | 7.62 × 10¹¹ | 9.92 × 10³ |
| 8 | 1 × 10⁸ | 1 | 89.7 ± 0.5 | 1.63 × 10¹² | 1.58 × 10⁴ |
| 16 | 1 × 10⁸ | 1 | 148.3 ± 1.2 | 4.37 × 10¹² | 5.95 × 10⁴ |
| 24 | 1 × 10⁸ | 1 | 75.7 ± 0.6 | 1.25 × 10¹³ | 1.46 × 10⁵ |
| 36 | 1 × 10⁸ | 1 | 82.9 ± 0.6 | 7.29 × 10¹² | 1.05 × 10⁵ |
| 48 | 1 × 10⁸ | 1 | 98.7 ± 0.3 | 6.08 × 10¹² | 6.79 × 10⁴ |

As shown in Table 5, 1×10⁸ adipose-derived stem cells obtained by expansion in Example 7 were mixed with physiological saline, and the concentration of extracellular vesicles reached the maximum at the standing time of 24 hours, then the concentration of extracellular vesicles decreased as the standing time was prolonged.

In addition, the adipose-derived stem cells obtained by the expansion in Example 7 were mixed with Dulbecco's phosphate-buffered saline (DPBS) or physiological saline in the proportions shown in Table 6, respectively, left standing at 2-10 C for 24 hours and then centrifuged at an acceleration rate of 300 g for 5 minutes, the supernatant was taken out to a new tube and centrifuged again at an acceleration rate of 4000 g for 20 minutes, and then the supernatant was taken out and filtered through a 0.22 μm filter (Merck Millipore, billerica, MA, USA) to remove large vesicles. Amicon Ultra-15 with a molecular weight over 100 kDa (Millipore) was used to remove free protein, then the supernatant containing the extracellular vesicles was obtained after centrifugation at an acceleration rate of 4000 g, the concentration and particle size of extracellular vesicles in the solution was analyzed, and the results are reported in Table 6.

TABLE 6

| | | | | Extracellular vesicles | | |
|---|---|---|---|---|---|---|
| | | | Saline | Particle | Concentrations | Concentrations |
| | Number of cells | DPBS (ml) | (ml) | size (nm) | (particles/ml) | (particles/ml) |
| The first | 7 × 10⁷ | 1 | 0 | 80.9 ± 0.6 | 8.595 × 10¹² | 1.23 × 10⁵ |
| group | | | | | | |
| The | 7 × 10⁷ | 1 | 0 | 92.7 ± 1.9 | 8.73 × 10¹² | 1.25 × 10⁵ |
| second | | | | | | |
| group | | | | | | |
| The | 1 × 10⁸ | 0 | 1 | 84.4 ± 0.5 | 1.20 × 10¹³ | 1.20 × 10⁵ |
| third | | | | | | |
| group | | | | | | |
| The | 1 × 10⁸ | 0 | 1 | 89.0 ± 0.7 | 1.16 × 10¹³ | 1.16 × 10⁵ |
| fourth | | | | | | |
| group | | | | | | |

As shown in Table 6, 7×10⁷ or 1×10⁸ adipose-derived stem cells obtained by expansion in Example 7 were mixed with Dulbecco's phosphate-buffered saline (DPBS) or physiological saline, and the extracellular vesicles were analyzed after standing in an environment of 2-10 C for 24 hours, as a result, it was found that all concentrations of extracellular vesicles of the adipose-derived stem cells obtained by expansion in Example 7 were 1.23×10⁵ particles/cell or more, regardless of the number of 7×10⁷ or 1×10⁸ adipose-derived stem cells in DPBS or saline.

### Analysis of Growth Factor

The adipose-derived stem cells obtained by expansion in Example 7 were mixed with physiological saline and left standing at 2-10° C. for 24 hours, and the amount of the growth factors was analyzed by MILLIPLEX® MAP MULIPLEX DETECTION (Merck Milliplex, Model: luminexMagpix analyzer), and the results are shown in Table 7.

TABLE 7

| Growth factor | Amount (pg/ml) |
|---|---|
| HGF | 3210.52 ± 679.25 |
| G-CSF | 295.37 ± 43.57 |
| Fractalkine | 57.9 ± 3.39 |
| IP-10 | 45.37 ± 3.56 |
| EGF | 8.15 ± 0.04 |
| IL-1α | 1.51 ± 0.57 |
| IL-1β | 3.19 ± 0.03 |
| IL-4 | 15.11 ± 0.05 |
| IL-5 | 0.86 ± 0.05 |
| IL-13 | 2.27 ± 0.13 |
| IFNγ | 1.59 + 0.09 |
| TGFα | 0.13 ± 0.03 |
| sCD40L (CD 154) | 12.43 ± 0.28 |

Growth factors have potential use in the treatment of stroke such as: hepatocyte growth factor (HGF) can prevent neuronal death and promote the key to neuronal survival via pro-angiogneic, anti-inflammatory and immune-modulatory mechanisms. HGF can act on neural stem cells to increase nerve neuroregeneration. HGF has the potential to protect cells from entering hypoxia induced programmed cell death, apoptosis. HGF protects the neurons loss of function after stroke, thereby enhancing the neurological function of a patient. HGF has anti-apoptotic effect, and plays an assisting role with adipose-derived stem cells and endogenous neural stem cells to further promote recovery after stroke. Granulocyte Colony-Stimulating Factor (G-CSF) enhances recovery after stroke through neuroprotective mechanisms or neural repair. Transforming growth factor alpha (TGFα) plays an important role in nerve cell proliferation and differentiation, stimulating astrocytes synthesis of nerve growth factor. In addition, TGFα is an important endogenous protective factor of white matter, which can improve long-term functional recovery after stroke. Chemokine ligand 1 (CX3CL1) has a neuroprotective effect on cerebral ischemic damage. CX3CL1 is an important messenger molecule that acts as microglial cells to reduce inflammation and damage of microglial cells during ischemic damage. Epidermal growth factor (EGF) is a potent mitogen that promotes migration, proliferation of endogenous neural progenitors cells and induces production of astrocytes and neurons from central nerve system (CNS) precursor cell to allow for repopulation of neurons lost after partial stroke. Interferon gamma (IFNγ) can activate proregenerative, promyelinating and anti-inflammation of mesenchymal stem cells, thereby increasing the effect of treating ischemic stroke. Interleukin-1α (IL-1α) has angiogenesis after cerebral stroke; administration of IL-1α in the acute phase of ischemic stroke significantly increases neuroprotection, and administration of IL-1α in the subacute phase of ischemic stroke enhances vascular density and potential neurogenesis around cerebral infarction. Interleukin-4 (IL-4) promotes the ability of learning and memory in normal brain, and plays an important role in the regulation of brain cleanup and repair after cerebral stroke when damaged neurons initiate endogenous defense mechanism to secrete IL-4. Interleukin-5 (IL-5) plays a key role in atheroprotective immune pathway. Interleukin-13 (IL-13) can improve long-term neurological deficits and white matter damage caused by stroke; IL-13 plays a key role in the regulation of inflammatory and immune responses via the reverse regulation of pro-inflammatory factors production in microglia.

### Example 10 (Pharmaceutical Composition of the Invention)

The adipose-derived stem cells obtained by expansion in Example 7 were mixed with Dulbecco's phosphate buffered saline (DPBS) or physiological saline, and standing for 1, 2, 4, 8, 16, 24, 36 and 48 hours respectively in an environment of 2-10° C.; as described above, the preparation consisting of the adipose-derived stem cells and the extracellular vesicles and growth factors generated at a specific standing time is a pharmaceutical composition of the present invention (hereinafter referred to as TS pharmaceutical composition), which can be used for treating cerebral stroke.

In addition, the adipose-derived stem cells may also be standing in other water for injection to generate extracellular vesicles and growth factors, for example, the water for injection can be selected from distilled water for injection, 0.45%˜3% sodium chloride injection, 2.5%-50% glucose injection, Lactated Ringer's B injection, or Ringer's Solution, without limitation herein.

### Example 11

The present study (ClinicalTrials.gov Identifier: NCT02813512, NCT04088149) was in accordance with the ethical principles of the Declaration of Helsinki and local laws and regulations. The present study followed the current guidelines of the pharmaceutical good clinical trials industry.

The selective conditions for the subjects were as follows:

Inclusion Condition
(1) Your age is between 65 and 85 years.
(2) During the selection visit, it was 6 months to 15 years after your stroke occurred.
(3) You had suffered a stroke in the distribution area of the carotid artery. The site of stroke should be diagnosed via Magnetic Resonance Imaging (MRI).
(4) According to MRI assessment, the area of your brain damage was between 0.5 cm and 10 cm in diameter.
(5) The score of your National Institutes of Health Stroke Scale (NIHSS) during the selection visit was between 8 and 30.
(6) If you have a stroke with hemiplegia, your score of the fifth or sixth question of NIHSS will be less than 4 (for the affected limb) during the selection visit.
(7) Your score (±3) of NIHSS from selection visit (the first visit) to the second visit (before adipose tissue was collected) was stable for at least 2 weeks.
(8) Your Systolic pressure was less than 200 mmHg (should be the average of at least 2 measurements) before administration in the selection visit, the second visit (before adipose tissue was collected) and the third visit.
(9) Your international normalized ratio (INR) during the selection visit is less than 2.5, and the number of platelets is between 100,000 and 500,000 per microliter (1×10\hat{ }5/μL˜5×10\hat{ }5/μL).
(10) If you are a female subject and have fertility, it should be confirmed that you are not pregnant or breastfeeding from the selection period to the trial period.
(11) If you are a male or female subject with fertility (between puberty and 2 years after menopause), an effective and credible method of contraception, such as tubal ligation, vasectomy, intrauterine contraceptive device, intrauterine administration system, hormonal contraceptive or condom, should be used during the trial.
(12) The neurologist determines that your recent symptoms are related to the area of stroke.
(13) You or your legal representative are willing to sign the Informed Consent Form.

Exclusion Condition
(1) You have clinically significant autoimmune diseases such as rheumatoid arthritis, systemic lupus erythematosus, multiple sclerosis or psoriasis.
(2) You cannot perform MRI and computed tomography (CT) examination for any reason.
(3) You have multiple significant intracranial vascular stenoses (stenosis degree of more than 50%).
(4) You cannot temporarily stop treatment with antiplatelets [e.g. Aspirin and Persantin] and/or anticoagulants [e.g. Warfarin] within 3 days prior to your administration of autologous adipose-derived stem cells at the third visit.
(5) You have received systemic immunosuppressive therapy, immunotherapy or cytotoxic drugs within 1 month prior to the trial or during the selection visit.
(6) Your liver function deficiency during the selection visit: ALT (Alanine transaminase), AST (Aspartate transaminase) and ALP (Alkaline phosphatase) were above 2 times the upper limit of normal.
(7) Your renal insufficiency during the selection visit: blood urea nitrogen is 30 mg or more per deciliter (BUN≥30 mg/dl) or serum creatinine is 3 mg or more per deciliter (creatinine≥3 mg/dl).
(8) You have or are currently suffering from the following diseases: spinal cord injury, Alzheimer's disease, Parkinson's disease, spinocerebellar ataxia (cerebellar atrophy), spinal muscular atrophy, or other clinically significant neurological disorders that affect the assessment of this trial.
(9) You have a clinically serious and/or life-threatening illness such as uncontrolled diabetes or malignancy.
(10) You are at high risk of the following infectious diseases: HIV, syphilis, or human transmissible spongiform encephalopathy such as Creutzfeldt-Jakob Disease.
(11) You cannot generate enough autologous adipose-derived stem cells before transplantation surgery in the third visit.
(12) You are female subject and are breastfeeding, or pregnant, or are about to be pregnant.
(13) You have known or may be allergic to autologous adipose-derived stem cells or excipients thereof.
(14) Chest X-rays and ECG show that you have other complications.
(15) You have participated in other clinical trials and received treatment within 4 weeks prior to the selection visit.
(16) At the discretion of the trail moderator, you are not eligible to participate in this trail.

In the example, adipose-derived stem cells were obtained from a subject via the method of Example 7 and expanded as follows:

2˜5 g of adipose tissue is collected from subcutaneous adipose in the abdominal wall by performing liposuction from a subject via abdominal surgery, and the operation time for adipose extraction is about 1 hour or less, and the wound is less than 1 cm. All donors provided informed consent form (ICF). Adipose tissue was placed in Ca²⁺/Mg²⁺ free phosphate buffer solution (PBS) and immediately transferred to the laboratory.

Human adipose tissue was removed from the medium for transport and placed in a culture plate, washed 3 to 4 times with Ca²⁺/Mg²⁺ free phosphate buffered solution (PBS), and cut into small pieces (volume about 1-3 mm3). The tissue was dissociated with 0.1-0.3% collagenase for 60 minutes at 36.5-38.5° C. After digestion of collagenase, cells and undigested tissue fragments were separated from the granules of stromal vascular fractions (SVF) by centrifugation at 500 g for 5-15 min at 20-25° C., and dissociated cells were collected and cultured at 36.5-38.5° C. in an incubator supplied with 5% CO₂. After 1-2 days of culture, the supernatant and fragments were removed from the culture to obtain primary adipose-derived stem cells.

Primary adipose-derived stem cells (see Table 9 for number of cells) were cultured in the Keratinocyte-SFM medium (Gibco) (the same medium composition as in Example 1) containing L-ascorbic acid 2-phosphate containing 1-100 mM of N-acetyl-L-cysteine (Sigma), 0.05-50 mM of L-ascorbic acid 2-phosphate (Sigma) in a culture plate (HYPERFlask, Corning) made of a material containing an oxygen-containing functional group proportion of 20% or more to eighty percent full (about 10-14 days, the number of cells is about 7×10⁷-3.146×10⁸), and the culture environment is in a cell incubator controlled at a temperature of 36.5-38.5° C. and with 5% carbon dioxide.

The quality test related to stem cells in this study is performed by a third-party accredited laboratory (Taiwan Accreditation Foundation (TAF), accreditation standard: ISO/IEC 17025, certification Number: 2800). Sterility test shall be evaluated by direct inoculation method on the basis of the sterility test method in Chinese Pharmacopoeia and USP43 and Sterility Tests; Gram stain is another rapid microbial detection test, wherein the staining method was used to distinguish whether it is Gram positive/negative bacteria. Mycoplasmas was evaluated by nucleic acid expansion based on the method described in Chinese Pharmacopoeia. Endotoxin was examined according to the bacterial endotoxin test method specified in Chinese Pharmacopoeia and USP43 and Bacterial Endotoxins Tests, and evaluated by dynamic colorimetric method. Cell surface markers of CD34, CD45, CD90 and CD105 (Becton Dickinson) were analyzed using a BD AccuriC6 flow cytometer (Becton Dickinson). Cell survival rate was assessed with a ADAM-MC™ Automatic Cell counter (Digital Bio, NanoEnTek Inc.).

The relevant standard of adipose-derived stem cells after expansion are shown in Table 8:

TABLE 8

| Indications | | Chronic cerebral stroke |
|---|---|---|
| Source of stem cells | | Adipose |
| Number of cells | | 1-2 × 10⁸ ± 20% adipose-derived |
| | | stem cells |
| Cell viability (%) | | >80 |
| Expression of cell surface | CD34 | <10 |
| antigen (%) | CD45 | <10 |
| | CD90 | >90 |
| | CD 105 | >90 |

Safety

| | |
|---|---|
| Microbiological examination | Not detected |
| Endotoxin test (EU/mL) | <0.06 |
| Mycoplasma test | Non-reactive |
| Excipient | 1-1.2 ml physiological saline |
| Conditions for storage and standing | at 2-10° C. for 1-24 hours |

The basic data of subjects, the sampled amount of abdominal adipose tissue, the number of cells of adipose-derived stem cells before and after expansion, the cell viability, the expression of cell surface antigen after expansion and the safety of adipose-derived stem cells (amount of aerobic and anaerobic bacteria, endotoxin and Mycoplasma) are recorded in Table 9.

TABLE 9

| Code of subject | 17B001 | 17B002 | 17B003 | 005-20B-004 |
|---|---|---|---|---|
| Age | 73 | 75 | 67 | 65 |
| Sex | man | man | woman | man |
| Time of illness (years) | 1.6 | 6 | 2.4 | 2.3 |
| NIHSS | 17 | 16 | 17 | 10 |
| Sampled amount of | 4.9 | 4.98 | 4.3412 | 4.0573 |

adipose tissue (g)

Before expansion

| | | | | |
|---|---|---|---|---|
| Number of cells (number) | 8.24 × 10⁵ | 1.77 × 10⁶ | 1.85 × 10⁶ | 2.19 × 10⁶ |
| Cell viability (%) | 81 | 83 | 92 | 78 |

After expansion

| Number of cells (number) | | 2.1 × 10⁸ | 2.151 × 10⁸ | 3.146 × 10⁸ | 2.8356 × 10⁸ |
|---|---|---|---|---|---|
| Cell viability (%) | | 96 | 96 | 95 | 96 |
| Expression | CD34 | 0.18 | 0.62 | 0.3 | 0.07 |
| level of | CD45 | 0.46 | 0.25 | 0.25 | 0.08 |
| cell surface | CD90 | 100.0 | 99.99 | 100.0 | 99.98 |
| antigen (%) | CD 105 | 93.36 | 95.89 | 99.67 | 96.98 |

Safety

| | | | | |
|---|---|---|---|---|
| Bacteria | Not detected | Not detected | Not detected | Not detected |
| Endotoxin | <0.06 | <0.06 | <0.06 | <0.06 |
| Mycoplasma | Non-reactive | Non-reactive | Non-reactive | Non-reactive |

The adipose-derived stem cells obtained by expansion of the above-mentioned subject (hereinafter referred to as TS stem cells) were mixed with 1-1.2 ml of physiological saline, and extracellular vesicles were stored in an environment of 2-10° C. and standing for 24 hours, thereby obtaining a pharmaceutical composition containing TS stem cells, extracellular vesicles and growth factors (hereinafter referred to as a TS pharmaceutical composition); wherein subjects 17B001, 17B002 and 17B003 used 1×10⁸±20% TS stem cells, and subjects 005-20B-004 used 2×10⁸±20% TS stem cells.

As shown in Table 9, all the subjects were diagnosed with chronic stroke and the scores of NIHSS before treatment were 17, 16, 17 and 10, and were judged as moderate to severe stroke, which meet the conditions of inclusion and exclusion in the clinical trial and entered into this clinical trial. Prior to treatment, the subject is arranged for brain computed tomography (CT) and magnetic resonance imaging (MRI) scan, and the both kinds of images are combined to determine and label three injection points along corticospinal tract and near the area of infarction of the subject. Next, the subject was shaved around the surgical site of the subject under general anesthesia, and a high-speed surgical drill (Midas Rex MR7 High-Speed) was used to drill a hole at the marked site, and then the TS pharmaceutical composition obtained by standing adipose-derived stem cells as described above was injected into the subject at three injection points. Immediately after completion of the injection, a CT scan was performed to check the injection site and whether intracranial hemorrhage occurred. If no safety issues happened, the subject was returned to the rehabilitation room or general ward and was hospitalized for three days to monitor for safety issues.

Upon confirmation of no safety concerns, subjects were arranged for changes assessed by the National Institutes of Health Stroke Assessment Scale (NIHSS), the Barthel Index Scale (Barthel Index), the Berg Balance Test, the Fugl-Meyer Assessment (FMA), the Grip Strength Test, and the Purdue Pegboard Test (PPT) within 200 days after injection.

National Institutes of Health Stroke Scale (NIHSS), a standardized neurological examination scale designed for ischemic stroke is used to assess disease severity and consists of 15 items, including level of disturbance of consciousness, ability to answer questions, ability to follow commands, gaze, visual, facial palsy, motor left arm, motor right arm, motor left leg, motor right leg, limb ataxia, sensory function, language, dysarthria, sensory neglect; each item was scored on a scale of 3 to 5, ranging from 0 to 42. When summed, scores can be divided into 0 for normal, 1-4 for minor stroke, 5-15 for moderate stroke, 16-20 for moderate to severe stroke, and 21 or more for severe stroke, and a higher score indicates more severe nerve damage.

The Barthel Index is an assessment scale of daily living function, which consists of 10 items: feeding, transfers, grooming, toilet use, bathing, mobility (on level surfaces), stairs, dressing, bladder or bowel control, and the score range is 0-100, the total score can be divided into 0-20 completely dependent, 21-60 severely dependent, 61-90 moderately dependent, 91-99 mildly dependent, 100 completely independent, and a higher score thereof represents higher autonomous ability of a patient.

Balance is an important basis for the independence of function of daily life, and sensory and motor functions of a patient after cerebral stroke are weakened or lost, resulting in different degrees of balance disorders, thus affecting the independence of daily life, in this study, Berg Balance Test and FMA exercise scale were used to evaluate balance of a patient.

Berg Balance Test contains 14 daily life test items such as: maintaining sitting posture, standing to sitting, transfer (chair), sitting to standing, standing unsupported, standing with eyes closed, turning while standing, picking up object from the floor from a standing position, reaching forward while standing, standing with feet together, standing unsupported one foot in front, etc., and each has 5-grade score (0-4 points) ranging from 0-56 points, and a higher score thereof represents better balance function of a patient.

Fugl-Meyer Assessment (FMA) includes sensation (FMAS) and motor (FMAM) assessments. FMAM is used to measure upper limb movement and contains 33 items with scores ranging from 0 to 66, and higher scores thereof represents better upper limb motor function of a patient. Scores of FMAS range from 0 to 44, and a higher score thereof represents better sensory of a patient.

The grip strength test was considered to be associated with frailty, with a hand grip strength ≤30 kg for males and ≤20 kg for females as an indicator of impending rapid frailty.

The Purdue pegboard test (PPT) was used to assess mobility of one and two hands, finger, fingertip and arm mobility, and a higher score thereof represents better mobility of a patient.

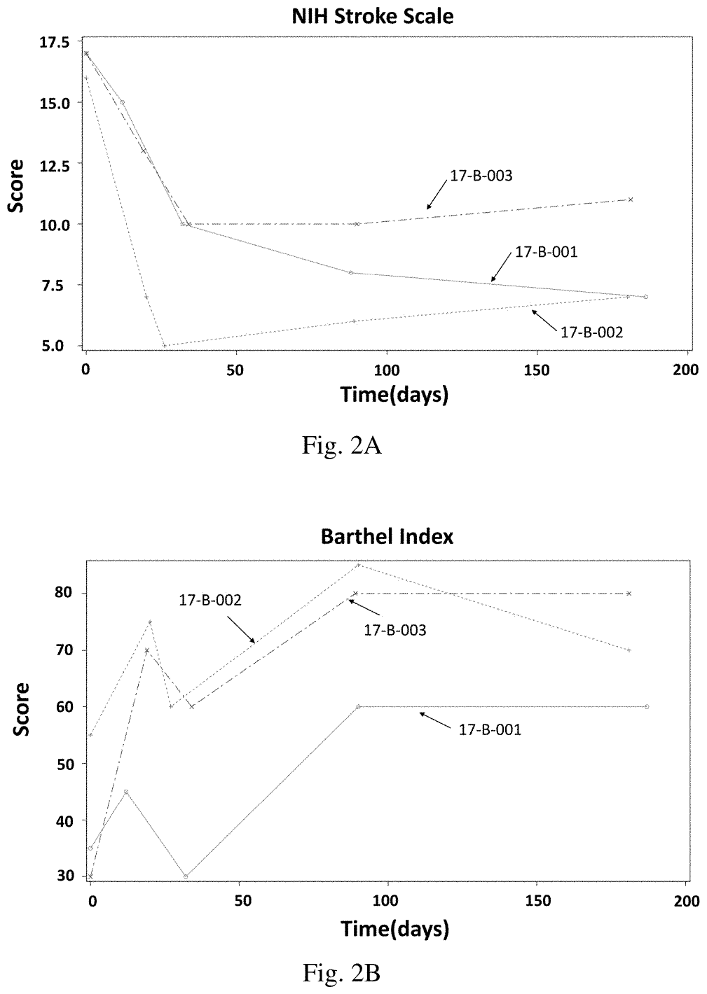

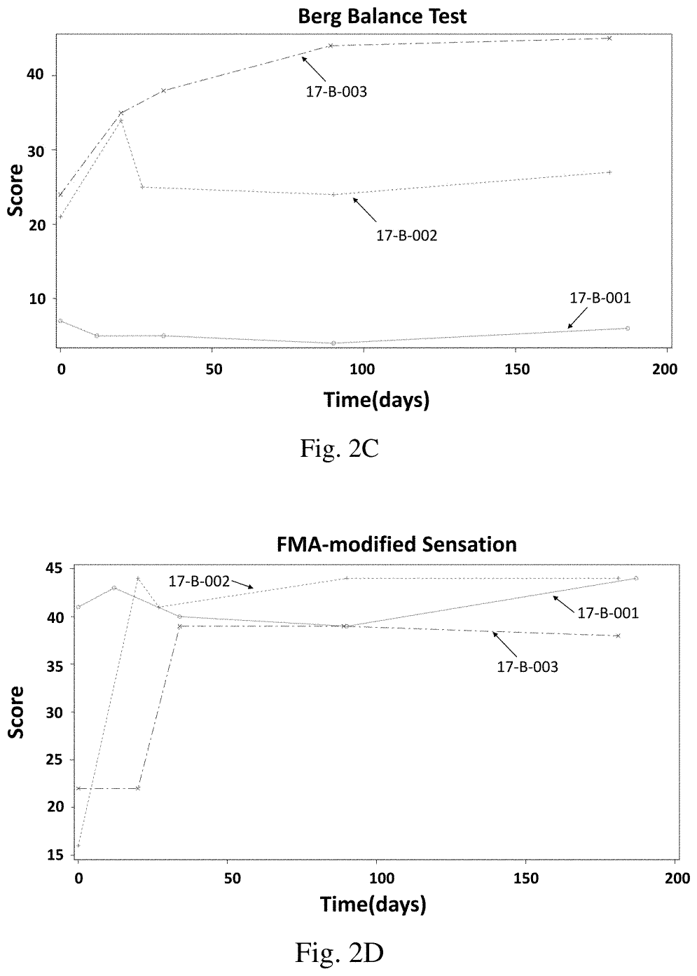

Referring to FIGS. 2A-2D, there are graphs sequentially showing changes in NIHSS, Barthel Index assessment, Berg Balance Test, and Fugl-Meyer assessment (FMA) of subjects 17B001, 17B002, and 17B003 sequentially within 200 days after injection of a TS pharmaceutical composition.

It can be seen from the results in FIG. 2A that the scores of NIHSS of subjects 17B001, 17B002 and 17B003 significantly decreased from 16 to 20, and the decrease was most obvious in the first 50 days, about 5-10, and then slightly increased gently; as can be seen from the results in FIG. 2B, the scores of Barthel Index of subjects 17B001, 17B002, and 17B003 showed an upward trend within 200 days after injection, which indicates that injection of a TS pharmaceutical composition can improve daily living activities of a subject.

Also, as can be seen from the results in FIG. 2C, the score of subject 17B003 increased from 24 to 45 while the score of subject 17B002 slightly increased from 21 to 27 in aspects of the score of Berg Balance Test; however, the score of subject 17B001 remained low with no improvement. As can be seen from the results in FIG. 2D, the FMA score of subject 17B002 increased from 16 to 44, while the FMA score of subject 17B002 increased from 22 to 38; and subject 17B001 did not undergo any pre-treatment for sensory deficits and therefore had very limited improvement in the FMA score from 41 to 44.

Subsequently, in the part of the grip strength test, subject 17B003 after treatment had an increase in left hand from 15.73 kg to 26.5 kg; an increase in right hand from 1.1 kg to 3.07 kg, and the left hand improved significantly. Subject 17B001 and subject 17B002 had no significant change.

In the part of the Purdue Pegboard Test (PPT), subject 17B003 after treatment progressed from nearly 0 to about 11 points (left hand). Subject 17B001 and subject 17B002 had no significant change.

In addition, subjects 17B001, 17B002, and 17B003 were arranged for an somatosensory evoked potential test (SSEP) at each return visit, which showed that subject 17B001 had an evoked response at the fifth visit, subject 17B001 had an evoked response at the third visit, and subject 17B001 had an evoked response from the fourth visit to the sixth visit.

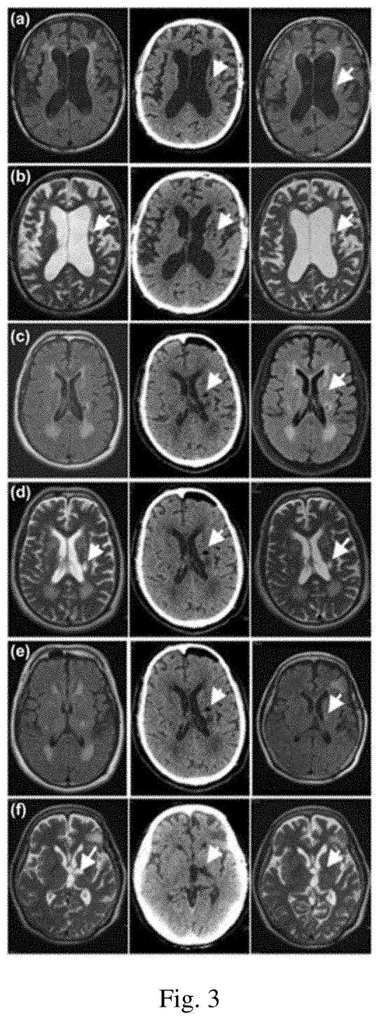

Furthermore, subjects 17B001, 17B002 and 17B003 were arranged for MRI scanning two weeks and six months after the injection of stem cells, respectively; with reference to FIG. 3, (a), and (b) are images of subject 17B001, (c), and (d) are images of subject 17B002, and (e), and (f) are images of subject 17B003. In the graph, the left column of (a), (c) and (e) shows the T2-FLAIR image before injection; the left column of (b), (d) and (f) shows T2 images before injection (white arrows indicate the area of infarction); the middle column of the graph shows the CT scan image detected immediately after injection (white arrow indicates one of the three injection sites); in the graph, the right column of (a), (c) and (e) shows the T2 image detected 2 weeks after injection; and the right column of (b), (d) and (f) shows the T2 images detected 6 months after injection.

As can be seen from the results in FIG. 3, when comparing the MR T2 images of pre-injection and 6-month post-injection, the infarct size was reduced in all subjects ((b), (d), and (f) in FIG. 3), and the area of infarction even almost completely disappeared in subject 17B002 ((f) in FIG. 3).

In addition, the score of NIHSS of a subject 005-20B-004 decreased from 10 to 9 within 200 days after injection of the TS pharmaceutical composition; the score of Barthel Index increased from about 85 to 100 within 50 days after injection (able to complete daily life independently); FMAM increased about 13 points after 200 days after injection; FMAS increased about 20 points after 200 days after injection; for the part of grip strength test, about 7 kg for the left hand and about 21 kg for the right hand can be gripped to about 42 kg to restore normal; and the part of the Purdue Pegboard Test (PPT) progressed from nearly 0 points to about 9 points (left hand+right hand+both hands).

It can be seen from the above-mentioned examples that autologous adipose-derived stem cells from a chronic stroke patient after being expanded and then injected into the brain of the patient can overcome the shortcomings of the previous intravenous injection that cannot effectively enter the brain due to the blood-brain barrier, thereby effectively improving the brain signal change and nervous system of the patient, and it can be found through multiple scales that the patient has significant improvement after 6 months, thus having great clinical value.

In view of the above, the present invention has been described with reference to the above examples, but the present invention is not limited to the embodiments. Those skilled in the art with common knowledge can make various changes and modifications without departing from the spirit and scope of the invention; for example, the technical contents exemplified in the above examples are combined or changed to new embodiments, and these embodiments are also regarded as one of the contents of the present invention. Accordingly, the scope of protection sought in this application also includes the scope of the following patent applications and their definitions.

What is claimed is:

1. A pharmaceutical composition for treating chronic stroke, which is injected via brain into the cranium of a patient having chronic stroke for six months or more; the pharmaceutical composition is a suspension at least comprising TS stem cells, an active synergistic component and a growth factor, and wherein
the expression level of CD34 and CD45 of the TS stem cells is 10% or less, and the expression level of CD90 and CD105 is 90% or more;
the active synergistic component is an extracellular vesicle;
the growth factor is at least one selected from the group consisting of HGF, G-CSF, Fractalkine, IP-10, EGF, IL-1α, IL-1β, IL-4, IL-5, IL-13, IFNγ, TGFα and sCD40L; and
in the pharmaceutical composition, the amount of the TS stem cells is at least 1×10⁷/mL, the amount of the active synergistic component is 7×10¹¹˜1.5×10¹³/mL, and the amount of the growth factor is 0.01˜4,000 pg/mL.

2. The pharmaceutical composition for treating chronic stroke as claimed in claim 1, wherein the patient has a score of National Institutes of Health Stroke Scale (NIHSS) between 8 and 30.

3. The pharmaceutical composition for treating chronic stroke as claimed in claim 1, wherein the TS stem cells are obtained by culturing adipose-derived stem cells in an expansion medium; and the expansion medium is Keratinocyte-SFM medium containing 1-100 mMof N-acetyl-L-cysteine, 0.05-50 mM of L-ascorbic acid 2-phosphate.

4. The pharmaceutical composition for treating chronic stroke as claimed in claim 3, wherein the expansion medium is placed on a culture plate made of a material containing at least 20% or more oxygen-containing functional groups.

5. The pharmaceutical composition for treating chronic stroke as claimed in claim 3, wherein the initial culture cell density of the adipose-derived stem cells is 5,000˜15,000 stem cells/cm².

6. The pharmaceutical composition for treating chronic stroke as claimed in claim 3, wherein the source of adipose-derived stem cells is autologous or allogeneic.

7. The pharmaceutical composition for treating chronic stroke as claimed in claim 1, wherein the pharmaceutical composition is obtained by allowing the TS stem cells to stand in water for injection at a temperature of 2-10° C. for 1-24 hours, and the TS stem cells release the active synergistic component and the growth factor during standing.

8. The pharmaceutical composition for treating chronic stroke as claimed in claim 1, wherein an endotoxin test result of the TS stem cells is less than 0.06 EU/mL.

9. The pharmaceutical composition for treating chronic stroke as claimed in claim 1, wherein a Mycoplasma test result of the TS stem cells is no reaction.

10. The pharmaceutical composition for treating chronic stroke as claimed in claim 1, wherein the TS stem cells have an activity of at least 80% or more.

11. The pharmaceutical composition for treating chronic stroke as claimed in claim 1, wherein the particle size of the active synergistic component is 30 nm˜1 μm.

12. The pharmaceutical composition for treating chronic stroke as claimed in claim 1, wherein the active synergistic component express ALIX, TSG101, CD9 and CD81.

13. The pharmaceutical composition for treating chronic stroke as claimed in claim 1, wherein the amount of HGF is 2,000˜4,000 pg/ml.

14. The pharmaceutical composition for treating chronic stroke as claimed in claim 1, wherein the amount of G-CSF is 200˜400 pg/ml.

15. The pharmaceutical composition for treating chronic stroke as claimed in claim 1, wherein the amount of TGFα is 0.01˜0.2 pg/ml.

16. The pharmaceutical composition for treating chronic stroke as claimed in claim 1, wherein the amount of IL-4 is 10˜20 pg/ml.

17. The pharmaceutical composition for treating chronic stroke as claimed in claim 1, wherein the amount of IL-13 is 2-3 pg/ml.
